(19) United States
(12) Patent Application Publication
(10) Pub. No.: US 2023/0031265 A1
(43) Pub. Date: Feb. 2, 2023

(54) DEVICES, METHODS AND COMPOSITIONS FOR APTAMER SCREENING

(71) Applicants: APLIFE BIOTECH CORPORATION, Wilmington, DE (US); ONSEJO NACIONAL DE IN- VESTIGACIONES CIENTIFICAS Y TECNICAS (CONICET), Buenos Aires (AR)

(72) Inventors: Tomas Guido ROZENBLUM, Buenos Aires (AR); Ivan Gustavo POLLITZER, Buenos Aires (AR)

(73) Assignees: APLIFE BIOTECH CORPORATION, Wilmington, DE (US); CONSEJO NACIONAL DE IN- VESTIGACIONES CIENTIFICAS Y TECNICAS (CONICET), Buenos Aires, DE (AR)

(21) Appl. No.: 17/819,051

(22) Filed: Aug. 11, 2022

Related U.S. Application Data
(63) Continuation-in-part of application No. PCT/US2021/018746, filed on Feb. 19, 2021.
(60) Provisional application No. 62/979,991, filed on Feb. 21, 2020.

(51) Int. Cl. G01N 33/543; G01N 33/53

(52) U.S. Cl. CPC G01N 33/5438; G01N 33/5308; C12N 15/115

(57) ABSTRACT

Provided herein are devices, methods and compositions useful in obtaining aptamers for biosensor probes. Such methods, devices and compositions are useful for novel clinical or companion drug diagnostic and personalized theranostic assays.

### CROSS-REFERENCE

This application is based on, and claims priority to, PCT Patent Application No.: PCT/US2021/018746, filed on Feb. 19, 2021; and U.S. Provisional Patent Application No. 62/979,991, filed on Feb. 21, 2020. The contents of the prior applications are hereby incorporated by reference herein in their entirety.

### SUMMARY

Provided herein are devices, methods and compositions useful for aptamer screening. The compositions described herein (e.g., aptamers), in most cases, are single stranded oligonucleotides or polypeptides, with the ability to bind to target proteins and other target ligands, while inhibiting the activity of the target. This ability to affect the activity of the target makes the compositions of the present disclosure attractive for therapeutic and diagnostic applications. In addition, the compositions described herein are, in some cases, engineered to change their conformation upon ligand binding, making them ideal for label-free analytical assays. The compositions disclosed herein, in some cases, are aptamers. In some embodiments, the aptamers are modified with ease and can be labeled with dyes and functional groups either to obtain a signal or for immobilization on solid supports. Aptamer activity is measured or modulated using the methods disclosed herein, by competitive interaction with a target molecule or hybridization with a complementary nucleotide sequence.

Despite recent advances in existing aptamer screening technology, there remains significant limitations. These limitations include (1) experimental statistical noise associated with chemical amplification techniques used in the systematic evolution of ligands by exponential enrichment (SELEX) and (2) the narrow variety of naturally occurring nucleotides of the genetic code. The lack of variety among naturally occurring nucleotides limits aptamer-target interactions and the efficiency of aptamer selection. Thus, there is a need for aptamer screening methods, systems and compositions with improved signal-to-noise ratios and that harness the expanded genetic code by utilizing modified nucleotides.

Disclosed herein, in some embodiments, are modified nucleotides comprising a modified nucleotide base, sugar and/or the sugar-phosphate backbone of aptamers, making it possible to generate hydrophobic and positively charged nucleotides via the addition of non-naturally occurring chemical functional groups. Additionally, the modified nucleotides of the present disclosure are used to circumvent the susceptibility of the aptamer to nuclease degradation. The modified nucleotides and aptamers are utilized by the biosensor devices, methods and compositions, described herein to provide, in some instances, for onsite, real time, label free sensing. Additionally, the aptamer-based devices, methods and compositions provided herein, in some instances, allow for screening aptamers as diagnostics and therapeutics.

Provided herein, in various instances are devices, methods and compositions comprising a substrate comprising one or more sensors; one or more probes attached to one or more sensors, wherein the one or more probes comprise: an aptamer; and one or more redox molecules; and an electrochemical circuit configured as a multiplexed amperometric biosensor device, wherein the one or more probes, electrochemical circuit and substrate comprise an integrated biosensors device. In some embodiments, the biosensor device comprises a substrate comprising a CMOS device. In some embodiments, the one or more sensors comprise working electrodes. In some embodiments, the aptamer comprises one or more nucleotides. In some embodiments, the nucleotides comprise modified nucleotides. In some embodiments, the aptamer specifically binds to the target. In some embodiments, target comprises a small molecule, peptide, protein, oligomer, or ligand that is present in the sample to be analyzed by the biosensor device.

In some embodiments, the electrochemical circuit comprises one or more working electrodes, one or more counter electrodes and none or one or more reference electrodes, operably connected to a multipotentiostat; wherein the electrochemical circuit is configured for amperometric measurements. In some embodiments the electrochemical circuit comprises one or more working electrodes, one or more counter electrodes and no reference electrode, operably connected to a multipotentiostat; wherein the electrochemical circuit is configured for amperometric measurements. In some embodiments, the CMOS device, comprises a first working electrode of the one or more working electrodes operably connected a first transimpedance amplifier of one or more transimpedance amplifiers, wherein the transimpedance amplifier is operably connected to an analog-to-digital converter (ADC). In some embodiments, the CMOS comprises one or more ADCs. In some embodiments, the working electrodes comprise gold.

In some embodiments, the working electrodes comprise hydrogenated amorphous carbon or the working electrodes comprise other materials having a surface with exposed OH groups disposed thereon. A gold surface alone does not necessarily have OH groups disposed thereon. However, such OH groups can be added to the gold surface with molecules such as diazonium salts like, for example, 4-carboxybenze diazo chloride.

Having OH groups disposed over the gold surface, or surface amorphous carbon or other materials with exposed OH groups disposed thereon, allows attaching covalently the probes (the detection element). The attachment of the probes to the exposed OH groups on the surface of materials (such as gold or amorphous carbon) are stronger than the attachments of the probes to just the gold surface or other surfaces without OH groups.

In another aspect are methods of detecting a target comprising contacting the one or more sensors with a sample comprising one or more targets; changing the electrical surface potential of the one or more sensors thereby generating one or more electrical current signals corresponding to the one or more sensors; and measuring the intensity of the one or more signals to detect the one or more targets. In some instances, the target comprises a small molecule. In some instances, the electrical current signal is generated by a change in the surface potential of a first working electrode of the one or more working electrodes due to a change in distance between the one or more redox molecules of a first probe of the one or more probes and the first working electrode caused by a change in the confirmation of the aptamer upon binding with the target. In some embodiments, redox molecule denotes a molecule capable of accepting or donating an electron thereby changing its redox state.

In some instances, the methods provided herein, can synthesize aptamer probes on a substrate. In some embodiments the methods comprise: (a) a printer being provided, the printer comprises a printhead, the printhead comprises one or more print nozzles, (b) a substrate being provided for printing on the substrate, (c) a droplet from a first print nozzle of the one or more print nozzles is printed to a first indexed location of the one or more indexed locations on the substrate; (d) replicating step (c) for a second print nozzle or more print nozzles; (e) washing the substrate; and (f) repeating step (c) through (e) one or more times. In some instances, the droplet comprises a nucleotide. In some instances, the droplet comprises a redox molecule.

In some embodiments, a probe composition has the formula: [[A]ₙ[X]m]y-L-S, wherein each A independently comprises a monomer linked to one or more redox molecules, each X independently comprises a monomer, L comprises a linker, S comprises a substrate, each n is independently an integer from 0 to 100, each m is independently an integer from 0 to 10, and y is an integer from 1 to 10. In some embodiments, the monomer of one or more A or X comprises a nucleotide. In some embodiments, the nucleotide comprises a modified nucleotide. In some embodiments, the linker comprises a thiol end group. In some embodiments, the substrate comprises gold. In some embodiments the substrate comprises hydrogenated amorphous carbon. In some embodiments the substrate comprises other materials that have exposed OH groups disposed on the surface of the material. In some embodiments, the one or more redox molecules comprise Ferrocene. In some embodiments, the one or more redox labels comprise Methyl Blue. In some embodiments, the probe comprises at least 3 redox molecules.

Having a substrate with a gold surface alone does not necessarily have OH groups disposed thereon. However, such OH groups can be added to the gold surface of the substrate with molecules such as diazonium salts like, for example, 4-carboxybenze diazo chloride.

Having OH groups disposed over the gold surface, or surface amorphous carbon or other materials with exposed OH groups disposed thereon, allows attaching covalently the probes (the detection element). The attachments of the probes to the exposed OH groups on the surface of materials (such as gold or amorphous carbon) are stronger than the attachments of the probes to just the gold surface or other surfaces without OH groups.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 exemplifies a device in accordance with an embodiment.

FIG. 2 exemplifies a method in accordance with an embodiment.

FIG. 3 exemplifies a device in accordance with an embodiment.

FIG. 4A exemplifies a device in accordance with an embodiment.

FIG. 4B exemplifies a device in accordance with an embodiment.

FIG. 4C exemplifies a device in accordance with an embodiment.

FIG. 4D exemplifies a device in accordance with an embodiment.

FIG. 4E exemplifies a device in accordance with an embodiment.

FIG. 5 exemplifies a device in accordance with an embodiment.

FIG. 6 exemplifies a device in accordance with an embodiment.

FIG. 7 exemplifies a method in accordance with an embodiment.

FIG. 8 exemplifies a method in accordance with an embodiment.

FIG. 9 exemplifies a method in accordance with an embodiment.

FIG. 10 exemplifies a method in accordance with an embodiment.

FIG. 11 exemplifies a method in accordance with an embodiment.

FIG. 12 exemplifies a method in accordance with an embodiment.

FIG. 13 exemplifies a method in accordance with an embodiment.

FIG. 14 exemplifies a method in accordance with an embodiment.

### DETAILED DESCRIPTION

Disclosed herein are methods, devices and compositions for aptamer discovery, which allows for the development of novel molecules for biosensor devices, diagnostic assays and therapeutics. Through practice of the disclosure herein, one achieves real-time, label free sensing with small devices compatible with point-of-care platforms, in some cases, without amplification bias and the intrinsic low chemical diversity of natural oligonucleotides found when practicing traditional SELEX. Additionally, disclosed herein is a method for synthesizing aptamer probes allowing for a highly controllable combinatorial chemistry capability. The flexibility of the high-throughput synthesis method allows for inclusion of labeling molecules that increase the sensitivity of the system into the probes. Thus, practice of some methods, devices and compositions for aptamer discovery consistent with the disclosure herein facilitates the broad application of biosensor analysis of samples, such as biological samples including small molecules, proteins, nucleic acids, among others.

The system, methods and compositions described herein allow for a flexible method for the rapid construction of aptamers DNA libraries on predefined locations over a Complimentary-Metal-Oxide-Semiconductor (CMOS) chip fabricated with materials that will allow real-time aptamer-ligands interaction measurements. Each sensor electrode is single element of the CMOS chip that can be functionalized with one type of aptamer probe. A single CMOS Chip can contain an array of N numbers of elements, being N up to thousands of elements. However, semiconductors having billions of elements have been described.

The technology will allow the miniaturization of the aptamer discovery process into aptamer arrays allowing better sensitivity and the high-throughput analysis of thousands or millions of molecules in parallel in a device of the size of a fingerprint. Even more, the technology, which works through transducing electrical signals, will open a new era in the healthcare digital products allowing the fabrication of assays compatible with any personal or mobile device.

An aptamer-based high-throughput platform for the discovery of bio-sensing molecules for biosensor devices capable of measuring and detecting a target molecule in real time, (ii) novel molecules for the treatment of human diseases, and (iii) aptamer chips capable to analyze the proteome of humans and other organisms.

In some embodiments, the integrated biosensor device includes: a substrate on which aptamer probes are synthesized, where the substrate consists of CMOS or PCB device. Additionally, the substrate may be made from glass or plastic. The substrate may contain a plurality of electrodes. Each electrode, or equivalently sensor may have a specific aptamer probe synthesized on it. In some embodiments the integrated biosensor device includes a multipotentiostat and software for analysis of the measured current, aptamer library design, aptamer results storage, or other analytical tools.

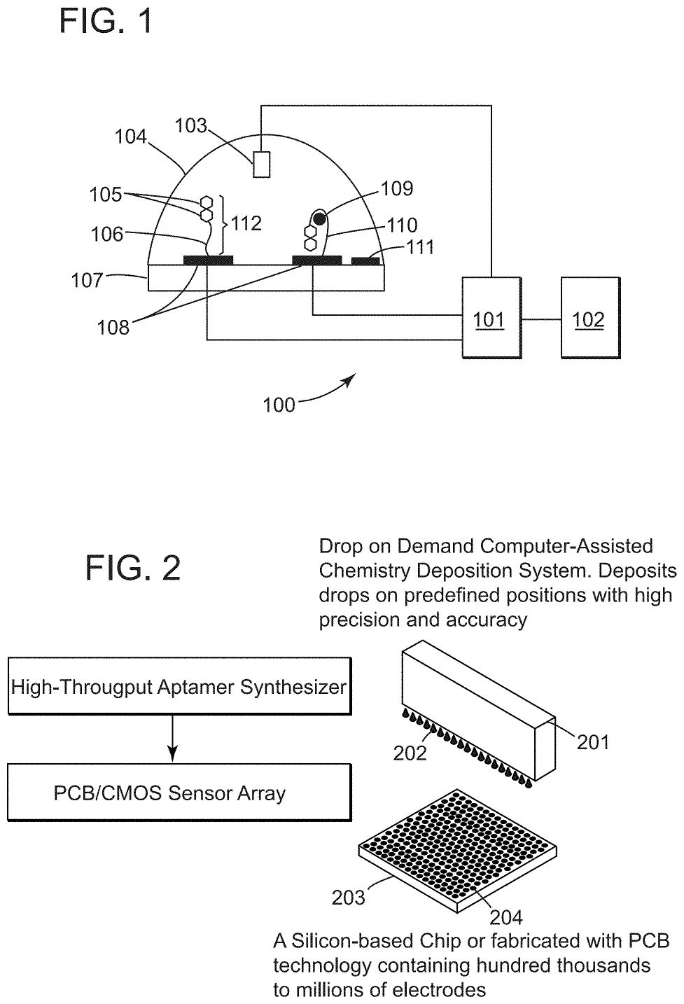

Referring to FIG. 1, an example of a biosensor device 100 is depicted, in accordance with aspects described herein. The biosensor device 100 includes a substrate 107, one or more probes 112 and an electrochemical circuit. The substrate 107 includes one or more sensors or working electrodes 108. The one or more probes 112 are coupled to the one or more sensors 108. The one or more probes 112 include an aptamer 106 and one or more redox molecules 105. The electrochemical circuit may include one or more counter electrodes 111 and optionally none, one or more reference electrodes 103. A buffer solution 104 may be disposed over the biosensor device 100 to provide a fluidic conductive path between the working electrodes 108 and counter electrodes 111.

The electrochemical circuit may be configured as an amperometric biosensor device, wherein the one or more working electrodes 108, one or more counter electrodes 111 and optionally none or one or more the reference electrodes 103 are operably connected to a multipotentiostat device 101, such that the electrochemical circuit is configured for amperometric measurements. The multipotentiostat device 101 may be connected to a computer 102 for further processing of the amperometric measurements.

In some embodiments, the biosensor device 100 is configured for amperometric sensing utilizing aptamer probes immobilized onto the working electrodes 108 and labeled with redox molecules 105 for current signal amplification, as seen in FIG. 1. In some embodiments, amperometric refers to a type of electrochemical sensor system where an electric potential is applied to the electrochemical cell and an electrical current resulting from either a reduction or oxidation reaction is measured. In some embodiments, working electrode refers to the electrode in an electrochemical sensor system, on which the sensing reaction occurs. The sensing reaction is between a probe, which is immobilized to the working electrode surface and a target, or analyte, to which the probe binds with specificity. If the reaction on the electrode is a reduction reaction, the working electrode is called cathodic. If the reaction on the electrode is an oxidation reaction the working electrode is called anodic. The substrate 107 may contain multiple working electrodes 108 which act as sensors. In some embodiments, the number of working electrodes is 1 to 10,000,000. In some embodiments, the number of working electrodes is 1 to 10, 1 to 100, 1 to 1,000, 1 to 10,000, 1 to 100,000, 1 to 1,000,000, 1 to 10,000,000, 10 to 100, 10 to 1,000, 10 to 10,000, 10 to 100,000, 10 to 1,000,000, 10 to 10,000,000, 100 to 1,000, 100 to 10,000, 100 to 100,000, 100 to 1,000,000, 100 to 10,000,000, 1,000 to 10,000, 1,000 to 100,000, 1,000 to 1,000,000, 1,000 to 10,000,000, 10,000 to 100,000, 10,000 to 1,000,000, 10,000 to 10,000,000, 100,000 to 1,000,000, 100,000 to 10,000,000, or 1,000,000 to 10,000,000. In some embodiments, the number of working electrodes is 1, 10, 100, 1,000, 10,000, 100,000, 1,000,000, or 10,000,000. In some embodiments, the number of working electrodes is at least 1, 10, 100, 1,000, 10,000, 100,000, or 1,000,000. In some embodiments, the number of working electrodes is at most 10, 100, 1,000, 10,000, 100,000, 1,000,000, or 10,000,000. In some embodiments, the width of the working electrodes is 1 micron to 10,000 microns. In some embodiments, the width of the working electrodes is 1 micron to 10 microns, 1 micron to 100 microns, 1 micron to 1,000 microns, 1 micron to 10,000 microns, 10 microns to 100 microns, 10 microns to 1,000 microns, 10 microns to 10,000 microns, 100 microns to 1,000 microns, 100 microns to 10,000 microns, or 1,000 microns to 10,000 microns. In some embodiments, the width of the working electrodes is 1 micron, 10 microns, 100 microns, 1,000 microns, or 10,000 microns. In some embodiments, the width of the working electrodes is at least 1 micron, 10 microns, 100 microns, or 1,000 microns. In some embodiments, the width of the working electrodes is at most 10 microns, 100 microns, 1,000 microns, or 10,000 microns. In some embodiments, the spacing of the working electrodes is 1 micron to 10,000 microns. In some embodiments, the spacing of the working electrodes is 1 micron to 10 microns, 1 micron to 100 microns, 1 micron to 1,000 microns, 1 micron to 10,000 microns, 10 microns to 100 microns, 10 microns to 1,000 microns, 10 microns to 10,000 microns, 100 microns to 1,000 microns, 100 microns to 10,000 microns, or 1,000 microns to 10,000 microns. In some embodiments, the spacing of the working electrodes is 1 micron, 10 microns, 100 microns, 1,000 microns, or 10,000 microns. In some embodiments, the spacing of the working electrodes is at least 1 micron, 10 microns, 100 microns, or 1,000 microns. In some embodiments, the spacing of the working electrodes is at most 10 microns, 100 microns, 1,000 microns, or 10,000 microns. Each working electrode 108 may be functionalized with an aptamer probe 106 that may be designed to bind specifically to a particular target molecule 109, act as a non-specific binding control, or to perform some other assay function.

The aptamer segment of the probe may be a specific nucleotide sequence, which may contain modified nucleotides. Additionally, the probe may contain one or more redox molecules such as Ferrocene or Methyl Blue, for example. In some embodiments, the number of redox molecules attached to one probe is 1 to 20. In some embodiments, the number of redox molecules attached to one probe is 1 to 2, 1 to 3, 1 to 4, 1 to 5, 1 to 10, 1 to 20, 2 to 3, 2 to 4, 2 to 5, 2 to 10, 2 to 20, 3 to 4, 3 to 5, 3 to 10, 3 to 20, 4 to 5, 4 to 10, 4 to 20, 5 to 10, 5 to 20, or 10 to 20. In some embodiments, the number of redox molecules attached to one probe is 1, 2, 3, 4, 5, 10, or 20. In some embodiments, the number of redox molecules attached to one probe is at least 1, 2, 3, 4, 5, or 10. In some embodiments, the number of redox molecules attached to one probe is at most 2, 3, 4, 5, 10, or 20. In some embodiments the counter electrode is off the substrate.

In some embodiments the counter electrode is fabricated onto the substrate, on the same surface as the working electrodes. In some embodiments, counter electrode refers to the electrode in an electrochemical system that functions as a cathode when the working electrode is operating as an anode. When the working electrode is operating as a cathode the counter electrode operates as an anode. The counter electrode can also be referred to as an auxiliary electrode.

The substrate may contain one or more counter electrodes 111. In some embodiments where the one or more counter electrodes are fabricated onto the substrate, the counter electrode may be designed to surround the working electrodes. In some embodiments where the counter electrode is fabricated onto the substrate, the counter electrodes may be interdigitated with the working electrodes.

In some embodiments, the biosensor device is contacted with a read buffer solution 104 that fluidically connects each probe functionalized working electrode 108 to one or more common reference electrodes 103 that are located off-substrate, as seen in FIG. 1, or used without a reference electrode. In some embodiments, reference electrode refers to the electrode in an electrochemical system that maintains a well-characterized electric potential and establishes the standard by which other electrode potentials are measured, specifically, the working electrode. In some embodiments, the working electrodes 108, the counter electrodes 111, and the reference electrode 103 are electrically connected to a multipotentiostat device 101, forming a circuit that is configured for amperometric detection.

The reference electrode 103 helps to compensate for the potential voltage drop between the counter electrode 111 and the working electrode 108 due to the resistivity of the liquid solution 104 that separates them. Hence, if the counter electrode 111 is placed close enough to the working electrode 111, the potential voltage drop becomes insignificant and the need for a reference electrode 103 is reduced or eliminated. For example, the need for a reference electrode 103 may be eliminated if the counter electrode 111 and working electrode 108 are within a range of 500 micrometers to 3 millimeters.

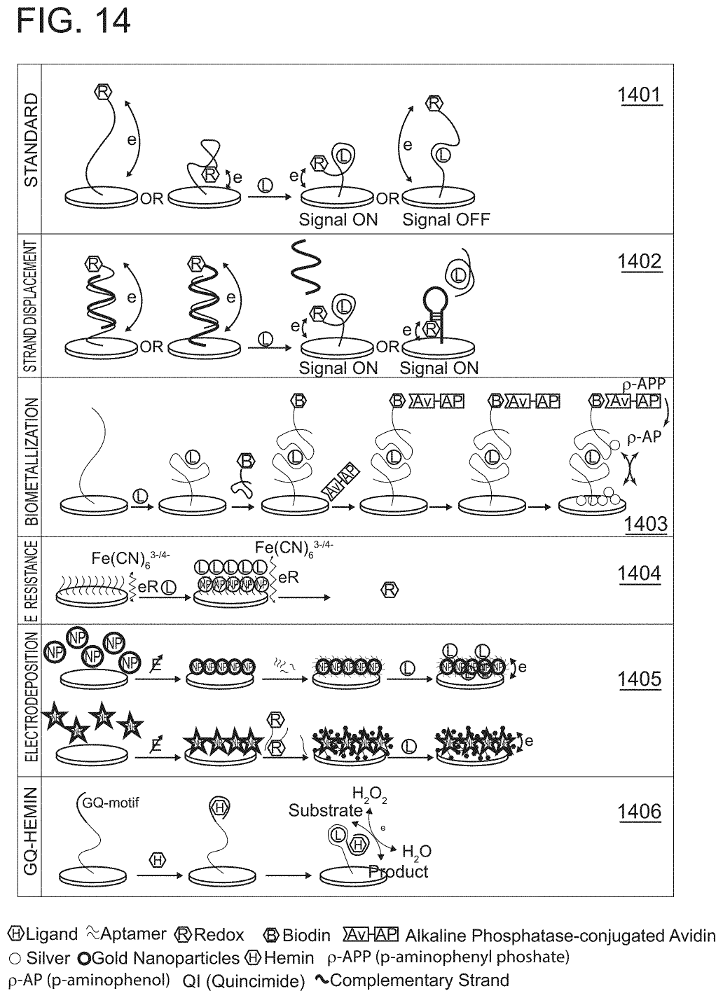

In some embodiments, potentiostat refers to an electronic device that controls the electric potential across an electrochemical circuit and measures the current. Potentiostats maintain the electric potential at the reference electrode with respect to the working electrode. This is done by increasing or decreasing the current supplied by the counter electrode. In some embodiments, multipotentiostat refers to a potentiostat capable of controlling multiple working electrodes. In some embodiments, the system is controlled by a computer 102. In some embodiments, a baseline electrical potential is established across the probe functionalized working electrodes 108 and a sample containing target molecules 109 is contacted to the surface of the array. In some embodiments, when a complimentary probe-target binding event occurs, the aptamer change in conformation 110, places the redox molecules 105 in closer proximity to the working electrode 108. When the redox molecules 105 move closer to the working electrode 108 surface, the electrical current increases. In other embodiments, when a complimentary probe-target binding event occurs, the aptamer change in conformation places the redox molecules 105 in further proximity to the working electrode 108 surface and the electrical current decreases, as seen in FIG. 14. These changes in electrical current, separately monitored for each working electrode, wherein the probe 106 is known to have been synthesized to contain a specific aptamer, indicate a hit between that aptamer and a target 109. In some embodiments, this process can occur in parallel across all working electrodes and allows for real-time, parallel molecular screening.

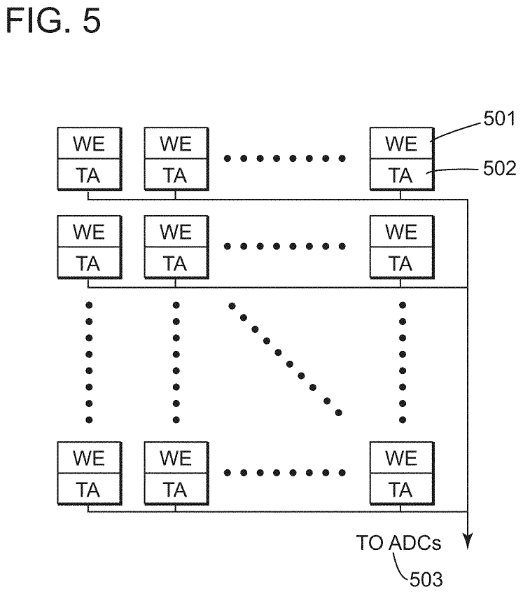

Referring to FIG. 5, an example of a matrix of working electrodes 501 is depicted, in accordance with aspects described herein. The matrix of working electrodes 501 may be included in a biosensor device as described herein, such as the biosensor device 100.

In some embodiments, the sensor array is a matrix of working electrodes 501, each with a direct connection to a transresistance amplifier 502, for signal conditioning as seen in FIG. 5. Every amplified signal is sent to an analog-to-digital converter 503, for digitizing. In some embodiments, a transimpedance amplifier is used as an alternative to a transresistance amplifier 502.

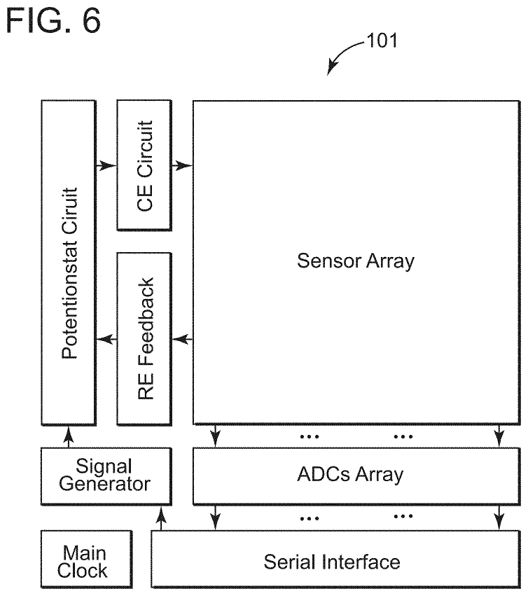

Referring to FIG. 6, an example of a multipotentiostat, such as multipotentiostat 101 of FIG. 1, is depicted, in accordance with aspects described herein. The multipotentiostat may be included in a biosensor device as described herein, such as the biosensor device 100.

In some embodiments, a multipotentiostat is used. FIG. 6. illustrates the basic function of the multipotentiostat. In this embodiment, the main clock synchronizes every other block of the device. The serial interface receives the instructions from a computer and, during the electrochemical procedure, sends the measured values back to the computer, for information processing. Through the serial Interface, the signal generator, makes the voltage signal for the potentiostat. The signal can be a continuous value, a triangle wave, square wave, or any combination of them that the test could require. The created signal reaches the potentiostat circuit. The potentiostat circuit stabilizes the sensors array potential, receiving information from the reference electrode feedback, and correcting the voltage error through the counter electrode circuit. The sensor array is the multi working electrode array, where the electrochemical process occurs, and the analog-to-digital converter, takes the information from the sensor array and digitizes it to send it through the serial interface, back to the computer, for further analysis.

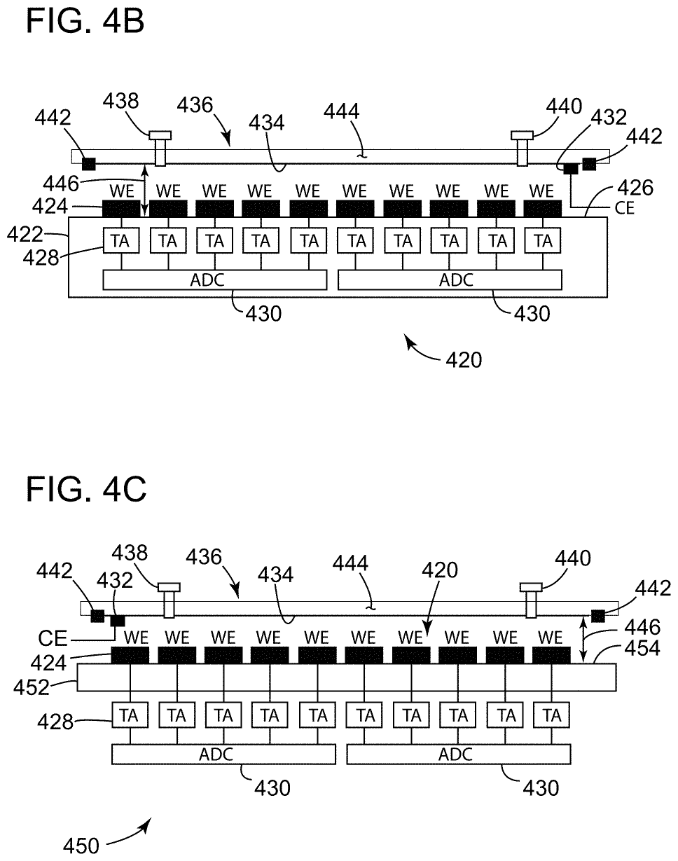

Referring to FIG. 3, an example of a CMOS device 300 is depicted, in accordance with aspects described herein. The CMOS device 300 may be included in a biosensor device as described herein, such as the biosensor device 100.

In some embodiments, a CMOS device 300 can be used as the substrate for the aptamer probe array as seen in FIG. 3. The working electrodes 303, which are the sensors in some embodiments, are located on the top surface of the device 300 and can be any conductive material. In some embodiments, the working electrode comprises 303. In some embodiments, where the substrate is a CMOS device, the working electrodes 303 are connected to the transimpedance amplifiers 302. In some embodiments, transimpedance amplifier refers to an amplifier that converts current to voltage and can be used to format the current output of a sensor as a readable signal. The transimpedance amplifiers may be connected in groups with an analog digital converter unit 301. The transimpedance amplifiers may be configured to condition the analog current signal prior to sending the current signal to the analog to digital converter. In some embodiments, the analog-to-digital converter is configured to convert the analog current signal to a digital signal and to send the digital signal out of the device for processing. A reference electrode 103 may be used, as seen in FIG. 1. In some embodiments, a CMOS device 300 is the substrate and the counter electrode 304 is fabricated onto the same plane as the working electrodes 303. and surrounds the array of working electrodes 303. In some embodiments, the counter electrode 304 is interdigitated amongst the working electrodes 303. In some embodiments, the electrical circuit comprises working electrodes, counter electrodes, a reference electrode and a multipotentiostat.

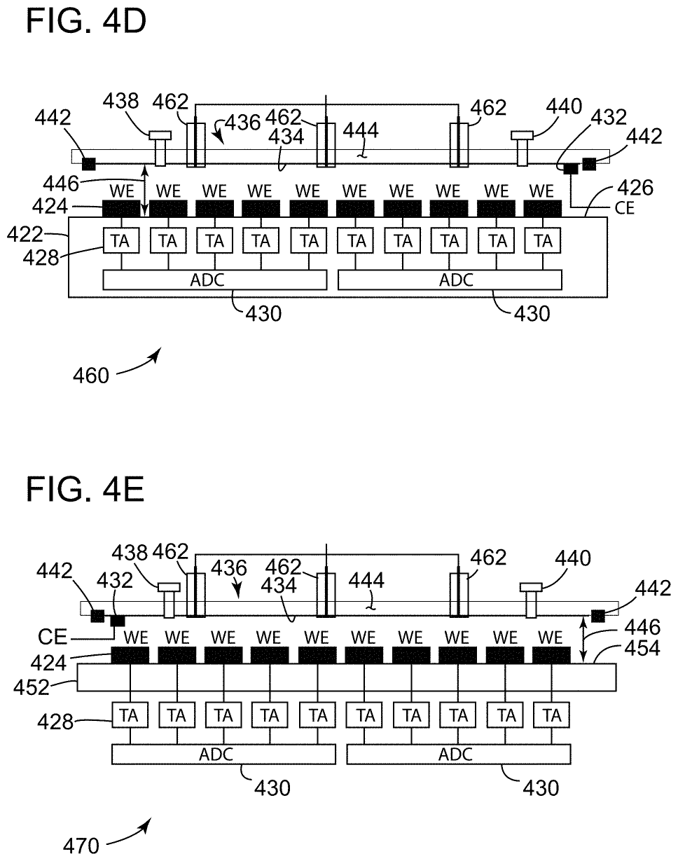

Referring to FIG. 4A, an example of a printed circuit board (PCB) device 400 is depicted, in accordance with aspects described herein. The PCB device 400 may be included in a biosensor device as described herein, such as the biosensor device 100.

In some embodiments, the biosensor device array can also be manufactured using PCB technology or printed or silk screened on various substrates 405 made of glass or plastic as seen in FIG. 4A. In some embodiments, the working electrodes 403 are connected to the transimpedance amplifiers 402, located off-substrate. In some embodiments, the transimpedance amplifiers are connected in groups to an analog-to-digital converter 401 that is also located off-substrate. In some embodiments, an off-substrate reference electrode 103 is used. In some embodiments, an on-substrate reference electrode is used.

In some embodiments, a transresistance amplifier is used in the biosensor device.

Referring to FIG. 4B, another example of a CMOS device 420 is depicted, in accordance with aspects described herein. The CMOS device 420 may be included in a biosensor device as described herein, such as the biosensor device 100.

CMOS device 420 is similar to that of CMOS device 300. CMOS device 420, like that of CMOS device 300, includes an array of working electrodes 424 positioned on the upper surface 426 of the substrate 422. The substrate 422 may be composed of glass, silicon, plastic or the like. The working electrodes 424 may be composed of any appropriate conductive material, such as, for example, tin, gold, copper, iron, tungsten or the like.

Each working electrode 424 of the array of working electrodes 424 is connected to an associated transimpedance amplifier 428. The transimpedance amplifiers 428 are connected in groups to one or more analog to digital converters (ADC) 430. The ADCs 430 sends data from the CMOS device 420 out to, for example, a multipotentiostat device 101 and then to a computer system 102 to be processed.

However, unlike CMOS device 300, a counter electrode 432 is positioned on the inner side 434 of a cover 444 of a lid 436 that encapsulates the CMOS device 420. The lid 436 includes an inlet port 438 and an outlet port 440 that are operable to allow a liquid solution (such as liquid buffer solution 104 of FIG. 1) containing analytes to be detected by the CMOS device 420 to pass through. The liquid passes over the surface 426 of the CMOS device 420 to deliver the analytes to the working electrodes 424, where they can be analyzed.

A gasket 442 extends around the perimeter of the cover 444 of the lid 436. The gasket 442 helps to prevent leakage of the liquid solution that is contained within the lid 436. Additionally, connections, such as wires or the like, that connect external peripheral devices (not shown) to the CMOS device 420, may pass through the gasket 442. The gasket 442 functions to prevent contact between the liquid solution and these connections.

The counter electrode 432 is preferrably positioned on the inner side 434 of the cover 444 of the lid 436 such that it is in contact with liquid solution, which provides a fluidic conductive path between the counter electrode 432 and the working electrodes 424. Additionally, by being in the inner side of the lid 436, the counter electrode 432 can be positioned close to the working electrodes 424. For example, the vertical space 446 between the counter electrode 442 and the working electrodes 424 may be within a range of 500 micrometers to 3 millimeters.

By positioning the counter electrode 432 and working electrodes 424 such that they are separated by a vertical space 446 within the range of 500 micrometers 3 millimeters, the potential voltage drop between the counter electrode 432 and the working electrode 424 due to the resistivity of the liquid solution that separates them is significantly reduced. Hence, if the counter electrode 432 is placed close enough to the working electrode 424, the potential voltage drop becomes insignificant and the need for a reference electrode to compensate for such a reduced potential voltage drop is reduced or eliminated. In the example shown in FIG. 4B, by being within the range of 500 micrometers to 3 millimeters, there is no need for a reference electrode. By eliminating the reference electrode, the cost of fabrication and complexity of CMOS device 420 is significantly and advantageously reduced.

Referring to FIG. 4C, another example of a PCB device 450 is depicted, in accordance with aspects described herein. The CMOS device 450 may be included in a biosensor device as described herein, such as the biosensor device 100.

The PCB device 450 is similar to the CMOS device 420 accept that the CMOS substrate 422 of CMOS device 420 is replaced by a printed circuit board (PCB) 452. Hence, all the functionally similar or like components in CMOS device 420 of FIG. 4B, that are also used in the PCB device 450 of FIG. 4C, are labeled with the same reference numbers in FIG. 4C. The printed circuit board 452 may be composed of glass or plastic. The working electrodes 424 are disposed on the upper surface 454 of the PCB 452.

Again, in PCB device 450, by positioning the counter electrode 442 close to the working electrodes 424, the need for a reference electrode may be reduced or eliminated. In the example illustrated in FIG. 4C, the counter electrode 442 is positioned on the inner side 434 of the cover 444 of the lid 436. The counter electrode 442 and working electrodes 424 are separated by a small vertical spacing 446, which is preferably within a range of 500 micrometers to 3 millimeters. By positioning the counter electrode 442 and working electrodes 424 so close together, the need for a reference electrode is eliminated.

Referring to FIG. 4D, another example of a CMOS device 460 is depicted, in accordance with aspects described herein. The CMOS device 460 may be included in a biosensor device as described herein, such as the biosensor device 100.

The CMOS device 460 is similar to the CMOS device 420 except that CMOS device 460 includes one or more reference electrodes 462 disposed on the inner side 434 of the cover 444 of the lid 436. Hence, all the functionally similar or like components in CMOS device 420 of FIG. 4B, that are also used in the PCB device 460 of FIG. 4D, are labeled with the same reference numbers in FIG. 4D.

In the example illustrated in FIG. 4D, the reference electrodes 462 extend through the cover 444 of the lid 436 to extend the sensor (or tip) end of the reference electrodes 462 just past the inner side 434 of the cover 444. The reference electrodes may (without limitation) include an internal element (such as, for example, silver-silver chloride), surrounded by an electrolyte-containing filling solution (such as, for example, KCl, saturated with AgCl), which is contained in either a glass or plastic body salt bridge, which terminates at a liquid junction. This liquid junction is made by press fitting a plug of teflon or other porous materials into the tip of the reference electrode. It is the tip of the reference electrode that extends past the inner side 434 of the cover 444 of the lid. Alternatively, and again without limitation, the reference electrode may include an Ag—AgCl electrically conductive ink as the electrolyte-containing filling solution.

The addition of the reference electrodes 462 helps to improve the signal from the ADCs 430 to the multipotentiostat device (or potentiostat) 101 by helping to compensate for the potential voltage drop between the counter electrode 432 and the working electrodes 424 due to the resistivity of the liquid solution (such as liquid buffer solution 104 of FIG. 1) that separates them. The counter electrode 432 and one or more reference electrodes 462 are advantageously on the inner side 434 of the lid 436 so that the liquid solution may provide a fluidic conductive path between the counter electrode 432, one or more reference electrodes 462 and working electrodes 424. It is also advantageous to have the counter electrode 432 and one or more reference electrodes 462 positioned close to the working electrodes 424. Preferably the counter electrode 432 and one or more working electrodes 462 are spaced a vertical distance 446 from the working electrodes 424 within a range of 500 micrometers to 3 millimeters.

If the one or more counter electrodes 462 include a plurality (that is two or more) of counter electrodes 462, then it is advantageous to electrically connect each reference electrode 462 in the plurality of reference electrodes 462 in parallel. This is because it is advantageous to obtain an average potential voltage drop between the counter electrode 432 and working electrodes 424 over the entire combined surface area that surrounds the working electrodes 424. By connecting each reference electrode 462 in the plurality of reference electrodes 462 electrically together in parallel and by positioning the reference electrodes over a large portion (for example 50 percent or greater) of the surface area containing or surrounding the working electrodes 424, the average potential voltage drop between the counter electrode 432 and working electrodes 424 is more closely obtained.

Referring to FIG. 4E, another example of a PCB device 470 is depicted, in accordance with aspects described herein. The PCB device 470 may be included in a biosensor device as described herein, such as the biosensor device 100.

The PCB device 470 is similar to the CMOS device 460 accept that the CMOS substrate 422 of CMOS device 460 is replaced by a printed circuit board 452. Hence, all the functionally similar or like components in CMOS device 460 of FIG. 4D, that are also used in the PCB device 470 of FIG. 4E, are labeled with the same reference numbers in FIG. 4E. The printed circuit board 452 may be composed of glass or plastic.

Again, the addition of the reference electrodes 462 helps to improve the signal from the ADCs 430 to the multipotentiostat device (or potentiostat) 101 by helping to compensate for the potential voltage drop between the counter electrode 432 and the working electrodes 424 due to the resistivity of the liquid solution (such as liquid buffer solution 104 of FIG. 1) that separates them. It is advantageous to have the counter electrode 432 and one or more reference electrodes 462 positioned close to the working electrodes 424. Preferably the counter electrode 432 and one or more working electrodes 462 are spaced a vertical distance 446 from the working electrodes 424 within a range of 500 micrometers to 3 millimeters.

If the one or more counter electrodes 462 include a plurality (that is two or more) of counter electrodes 462, then it is advantageous to electrically connect each reference electrode 462 in the plurality of reference electrodes 462 in parallel. This is because it is advantageous to obtain an average potential voltage drop between the counter electrode 432 and working electrodes 424 over the entire combined surface area that surrounds the working electrodes 424. By connecting each reference electrode 462 in the plurality of reference electrodes 462 electrically together in parallel and by positioning the reference electrodes over a large portion (for example 50 percent or greater) of the surface area containing or surrounding the working electrodes 424, the average potential voltage drop between the counter electrode 432 and working electrodes 424 is more closely obtained.

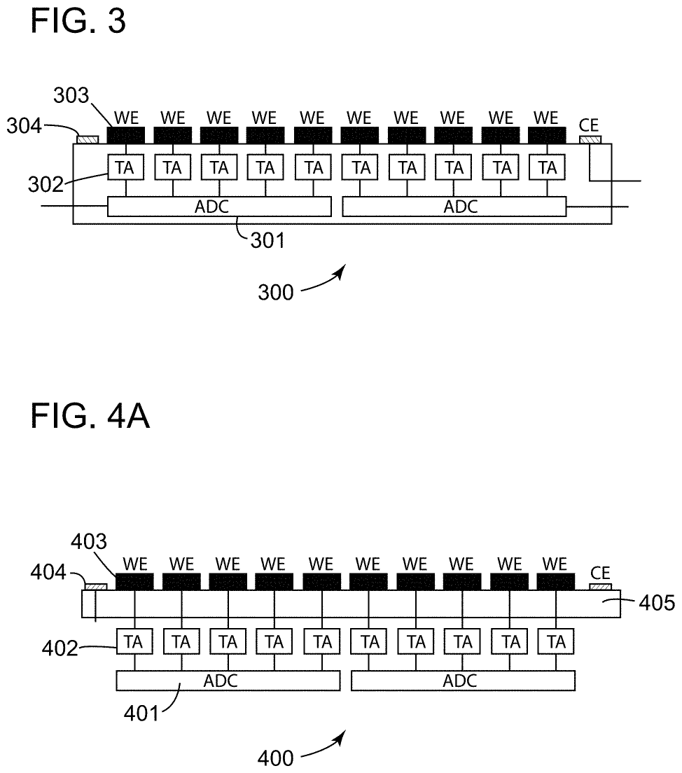

Referring to FIG. 2, an example of a Drop-On-Demand Computer-Assisted Chemistry Deposition System 200 is depicted, in accordance with aspects described herein. The system 200 may be included in one or more biosensor devices as described herein, such as the biosensor device 100.

In some embodiments, the biosensor device may consist of millions of probe types, where each type is defined by the probe's composition. In some embodiments, the number of probe types is 1 to 10,000,000. In some embodiments, the number of probe types is 1 to 10, 1 to 100, 1 to 1,000, 1 to 10,000, 1 to 100,000, 1 to 1,000,000, 1 to 10,000,000, 10 to 100, 10 to 1,000, 10 to 10,000, 10 to 100,000, 10 to 1,000,000, 10 to 10,000,000, 100 to 1,000, 100 to 10,000, 100 to 100,000, 100 to 1,000,000, 100 to 10,000,000, 1,000 to 10,000, 1,000 to 100,000, 1,000 to 1,000,000, 1,000 to 10,000,000, 10,000 to 100,000, 10,000 to 1,000,000, 10,000 to 10,000,000, 100,000 to 1,000,000, 100,000 to 10,000,000, or 1,000,000 to 10,000,000. In some embodiments, the number of probe types is 1, 10, 100, 1,000, 10,000, 100,000, 1,000,000, or 10,000,000. In some embodiments, the number of probe types is at least 1, 10, 100, 1,000, 10,000, 100,000, or 1,000,000. In some embodiments, the number of probe types is at most 10, 100, 1,000, 10,000, 100,000, 1,000,000, or 10,000,000. In some embodiments, each probe type is synthesized at pre-defined locations, corresponding to the working electrodes 108. In some embodiments the probes are synthesized onto the substrate at predefined locations, not including working electrodes. In some embodiments, the probes are synthesized on the device surface at high spatial resolution, using a piezoelectric ink-jet printhead. In some embodiments, the piezoelectric ink-jet printer is known as A Drop on Demand Computer-Assisted Chemistry Deposition System and is used to synthesize aptamer-based probes in predetermined, indexed positions on a planar surface, or substrate. Substrates may include complementary metal oxide semiconductor (CMOS) devices, printed circuit board (PCB) technology, glass and plastic. In some embodiments, the piezoelectric ink-jet printhead 201, containing multiple nozzles 202 can be used to print arrays 203 of modified aptamers and other molecules on arrays containing hundreds of thousands to millions of sensor elements 204 as seen in FIG. 2.

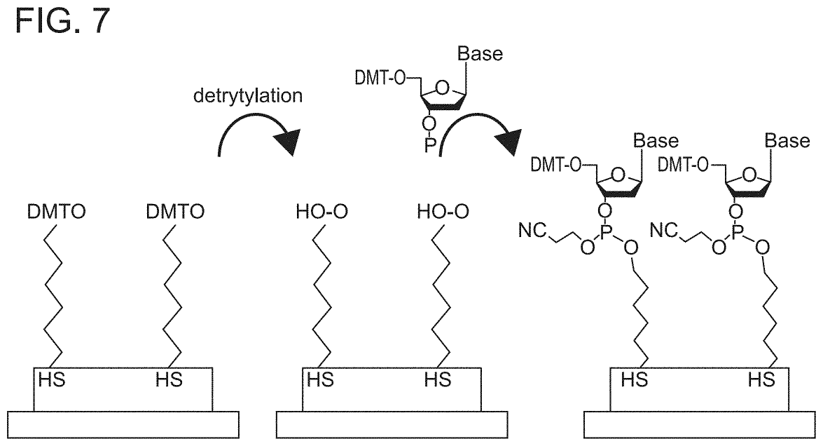

Referring to FIG. 7, an example a method of probe synthesis is depicted, in accordance with aspects described herein. The method of probe synthesis may be utilized in the formation of one or more aspects of a biosensor device as described herein, such as the biosensor device 100.

In some embodiments, probe synthesis is as following process: (1) a droplet containing a chemical linker with a reactive thiol end is deposited onto a gold electrode at an indexed location. This process is also repeated on all the electrodes other indexed locations. (2) After sufficient reaction time, the substrate is washed; and (3) a droplet containing a specific nucleotide, in some cases a modified nucleotide, or a nucleotide coupled to one or more redox molecules is deposited onto the linker functionalized electrode at the indexed location. This process is also repeated on all the electrodes at the other indexed locations. (4) After the sufficient reaction time, the substrate is washed. Steps (2) through (4) are repeated until the desired redox molecule labeled aptamer probes have been completely synthesized for each electrode at each indexed location on the substrate.

In some embodiments, synthesis is initiated over gold electrodes as seen in FIG. 7. The inkjet printer can be used to deliver droplets of synthesis reactants, individually, to each gold working electrode. The synthesis can be initiated by first coating the gold electrode with a chemical containing a thiol group, which anchors to the electrode, and a protective dimethoxytrityl (DMT) group in order to accept the phosphoroamidite group of the nucleotide bases in successive droplets. This substance for example can be 1-O-Dimethoxytrityl-hexyldisulfide, T-[(2-cyanoethyl)-(N,N-diisopropyl)]-phosphoramidite or another option can S-BZ-THIOL-MODIFIER C6-DT. This substance can be chemically reduced and chemi-adsorbed onto the gold electrodes. Then, the DMT group can be deblocked and a base with an activator can be added to react with the unprotected group. Following this initiation step, standard oligonucleotide synthesis is applied.

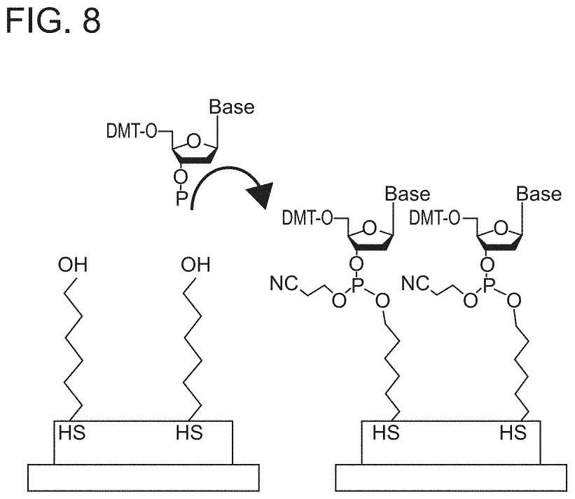

Referring to FIG. 8, another example a method of probe synthesis is depicted, in accordance with aspects described herein. The method of probe synthesis may be utilized in the formation of one or more aspects of a biosensor device as described herein, such as the biosensor device 100.

In some embodiments, as seen in FIG. 8, probe synthesis is initiated over gold electrodes, where the gold electrodes may be coated with a substance that contains a Thiol group for anchoring a hydroxyl group in order to accept any phosphoroamidite. This substance for example can be the alkanethiol 6-hydroxy-mercapto-hexanol. This substance can be chemically reduced and chemo-adsorbed onto the gold electrodes. Then, a base with an activator is added to react with the hydroxyl group. Following this initiation step, standard oligonucleotide synthesis is applied.

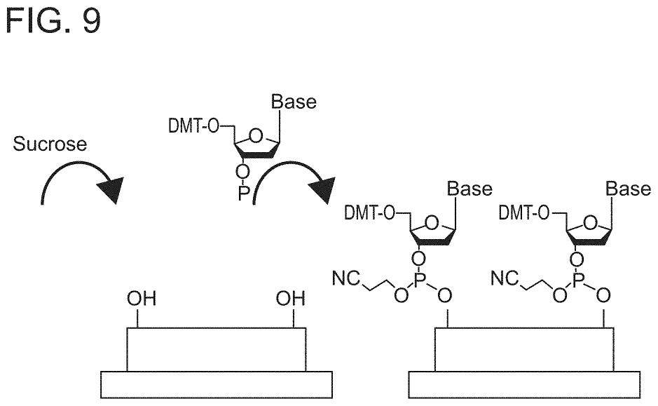

Referring to FIG. 9, another example a method of probe synthesis is depicted, in accordance with aspects described herein. The method of probe synthesis may be utilized in the formation of one or more aspects of a biosensor device as described herein, such as the biosensor device 100.

In some embodiments as seen in FIG. 9, synthesis is initiation over non-gold electrodes. To synthesize aptamer probes on non-gold, conductive electrodes, initiation can be carried out by coating the electrode with a substance that, after coating, adheres to the surface and leaves exposed hydroxyl groups. This substance for example can be the disaccharide sucrose. Then, a base with an activator can be added to react with the hydroxyl groups. Then, standard oligonucleotide synthesis can be applied.

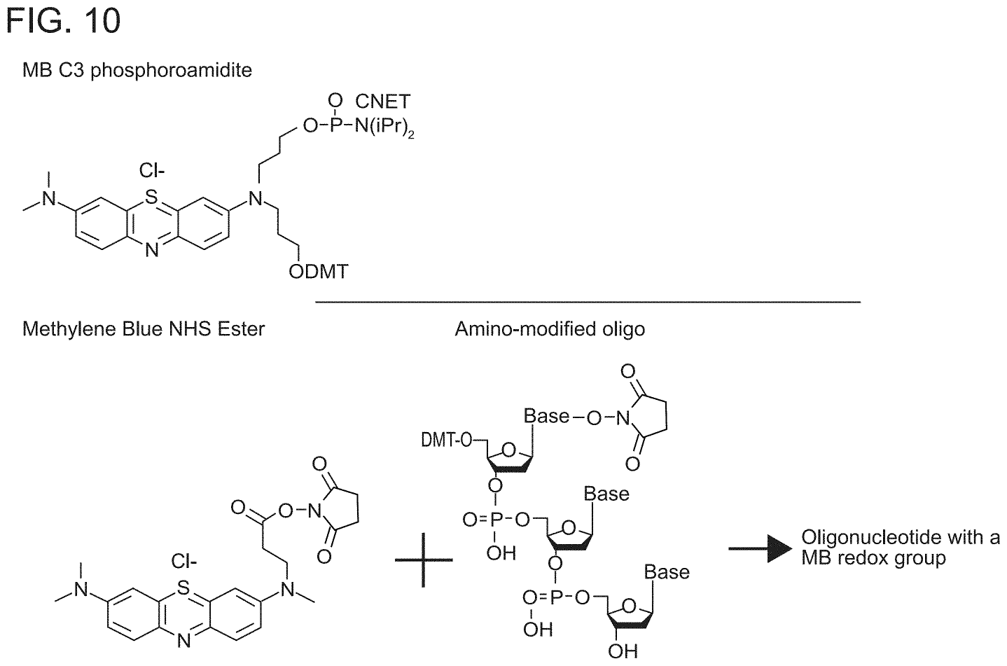

Referring to FIG. 10, an example a method of electrochemical detection with Methylene Blue is depicted, in accordance with aspects described herein. The method of electrochemical detection may be utilized in the detection of target molecules utilizing one or more aspects of a biosensor device as described herein, such as the biosensor device 100.

In some embodiments, electrochemical detection with Methylene Blue is achieved as seen in FIG. 10. A Redox group can be attached during oligonucleotide polymerization or post synthesis. For example, the Glen Research product MB C3 phosphoroamidite can be added during the synthesis, while Methylene Blue (MB) NHS, containing an amino accepting linker, can be added post synthesis to any amino modified nucleotide. Methylene Blue can be electrochemically reduced or oxidized using a potential range suitable for biological sensing.

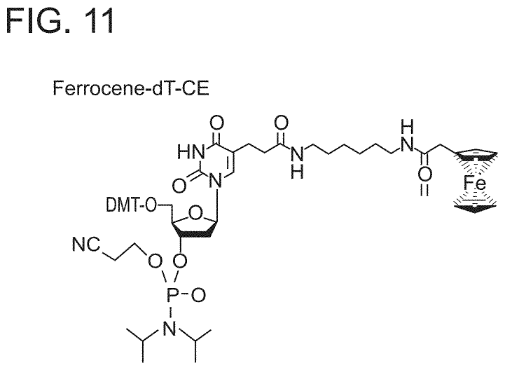

Referring to FIG. 11, another example a method of electrochemical detection with Ferrocene is depicted, in accordance with aspects described herein. The method of electrochemical detection may be utilized in the detection of target molecules utilizing one or more aspects of a biosensor device as described herein, such as the biosensor device 100.

In some embodiments, electrochemical detection with Ferrocene is achieved FIG. 11. A Redox group can be attached during oligonucleotide polymerization or post synthesis. For example, Ferrocene-dT-CE phosphoroamidite, can be added during the synthesis, while Ferrocene NHS, containing an amino accepting linker, can be added post synthesis to any amino modified nucleotide. Ferrocene can be electrochemically reduced or oxidized using a potential range suitable for biological sensing.

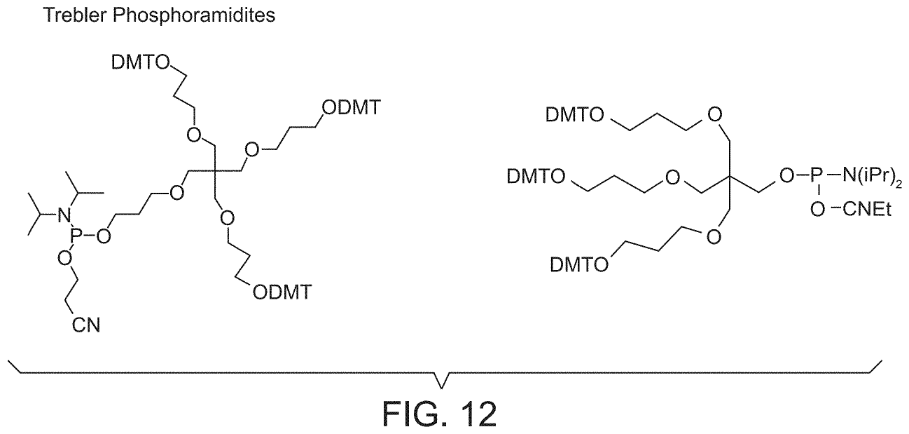

Referring to FIG. 12, an example a method of synthesis of aptamers is depicted, in accordance with aspects described herein. The method of synthesis of aptamers may be utilized in the formation of one or more aspects of a biosensor device as described herein, such as the biosensor device 100.

In some embodiments, synthesis of aptamers with enhanced redox molecules is achieved as seen in FIG. 12. Branching modification can be utilized to add several electrochemical redox molecules to one nucleic acid, aptamer probe. In order to increase the signal upon ligand binding, a branched phosphoramidite can be added during synthesis to increase the number of redox molecules in each probe molecule. In some embodiments, trebler phoshoramidites are used in order to add three redox amidites.

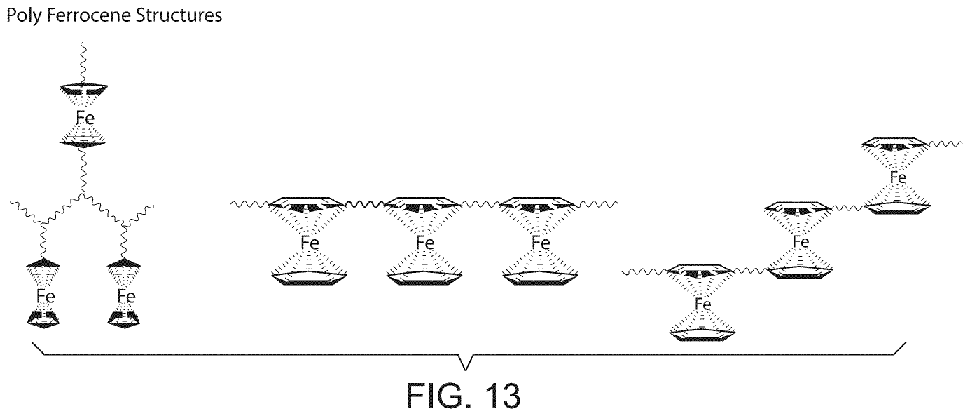

Referring to FIG. 13, another example a method of synthesis of aptamers is depicted, in accordance with aspects described herein. The method of synthesis of aptamers may be utilized in the formation of one or more aspects of a biosensor device as described herein, such as the biosensor device 100.

In some embodiments, synthesis of aptamers with enhanced redox reporters to enhance the signal upon target-ligand binding is achieved by adding several redox molecules, sequentially as seen in FIG. 13. In some embodiments, polyferrocene or polyMethyleneblue amidites are used in this manner.

Referring to FIG. 14, an example of various embodiments of methods designed to detect a ligand (or target molecule) is depicted, in accordance with aspects described herein. The various embodiments of methods may be utilized in the detection of target molecules utilizing one or more aspects of a biosensor device as described herein, such as the biosensor device 100.

In various embodiments, assays designed to detect a ligand electronically may include methods such as standard 1401, strand displacement 1402, biometallization 1403, electron resistance 1404, electrodeposition 1405 and GQ Hemin 1406, which are illustrated in FIG. 14, respectively. Some embodiments to detect a ligand electrochemically include utilizing Guanine (G)-rich stretches able to self-assemble into a secondary structure called G-quadruplex (GQ), monovalent cations, such as sodium and potassium, which play an important role in stabilizing GQ structures. In some embodiments, libraries can be designed to improve the binding of the aptamer probe to a ligand with GQ structures. GQ-based structures bound to a hemin molecule can be also used to improve the detection of aptamer-ligands Aptamer sequences such as this can be incorporated during library synthesis.

In some embodiments, a gold working electrode is functionalized with an aptamer probe, composed of a sequence of nucleotides, including modified nucleotides, and labeled with a sequence of 3 redox molecules. In some instances, the nucleotide sequence is attached to the gold surface of the working electrode by the reaction product of the linker 1-0-Dimethoxytrityl-hexyldisulfide,r-[(2-cyanoethyl)-(N,N-diisopropyl)]-phosphoramidite as seen in FIG. 7. In some instances, the nucleotide sequence consists of a 25 mer nucleotide sequence including modified nucleotides. In some embodiments, the nucleotide on the opposite end of the linker end, is labeled with a sequence of three Methyl Blue redox molecules.

In some embodiments, screening of biosensing aptamer molecules for electrochemical devices, screening of aptamers for fluorescence detection assays, screening of aptamers for enzymatic detection assays, engineering of existing aptamers to improve their performance, synthesis of oligo pools for synthetic gene development, synthesis of oligo pools for 3D DNA structures, synthesis of oligonucleotides for information storage, fabrication of DNA microarrays, all of the above using unlimited DNA modifications, and bias assays for CRISPR technology.

In some embodiments, an aptamer may be a nucleic acid molecule, such as RNA or DNA that is capable of binding to a specific molecule with high affinity and specificity. Exemplary ligands that bind to an aptamer include, without limitation, small molecules, such as drugs, metabolites, intermediates, cofactors, transition state analogs, ions, metals, nucleic acids, and toxins. Aptamers may also bind natural and synthetic polymers, including proteins, peptides, nucleic acids, polysaccharides, glycoproteins, hormones, receptors and cell surfaces such as cell walls and cell membranes. The binding of a ligand to an aptamer, which is typically RNA, causes a conformational change in the effector domain and alters its ability to interact with its target molecule. Therefore, ligand binding affects the effector domain's ability to mediate gene inactivation, transcription, translation, or otherwise interfere with the normal activity of the target gene or mRNA, for example.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

As used herein, the term ‘about’ a number refers to that number plus or minus 10% of that number. The term ‘about’ a range refers to that range minus 10% of its lowest value and plus 10% of its greatest value.

Further understanding of the disclosure herein is gained through reference to the following embodiments.

### EXAMPLES

### Example 1: Aptamer Screening Utilizing an Electrochemical Biosensor Device with Redox Amplification

A schematic diagram of an aptamer-based electrochemical biosensor device with redox amplification 100 is shown in FIG. 1, where a substrate 107 containing multiple working electrodes 108 as sensors is provided. Each working electrode 108 is functionalized with an aptamer probe 106 designed to bind specifically to a particular target molecule 109, act as a non-specific binding control, or other perform some other assay function. The aptamer segment of the probe is a specific nucleotide sequence, which may contain modified nucleotides. Additionally, the probe can contain one or more redox molecules such as Ferrocene or Methyl Blue, for example. The probe functionalized substrate contains counter electrodes 111 in addition to the probe functionalized working electrode's 108. The device can then be contacted with a read buffer solution 104 that fluidically connects each probe functionalized working electrode 108 to a common reference electrode 103. The multitude of working electrodes 108, the counter electrodes 111, and the reference electrode 103 are electrically connected to a multipotentiostat device 101, forming a circuit that is configured for amperometric detection. The entire system is controlled by a computer 102. A baseline electrical potential is established across the probe functionalized working electrodes 108 and a sample containing target molecules 109 is contacted to the surface the array. For this particular assay, the complimentary probe-target binding, causing the aptamer to change conformation 110, places the redox molecules in closer proximity to the working electrode 108. This decrease in distance between the redox molecules and the working electrode causes an increase in the electrical current, which is separately monitored for each working electrode known to have been synthesized with a specific aptamer. This electrical current change, separately monitored for each individual working electrode, acts as a signal indicating a hit between the aptamer and the target. Alternatively, the assay can be configured to allow the redox molecules to move away from the working electrode surface upon a change in conformation of the aptamer when the target binds, also causing a change in electrical current, separately monitored for each working electrode. This process can occur in parallel across all working electrodes and allows for real-time, label-free target, parallel molecular screening.

### Example 2: Method of Synthesizing Aptamer Probes on a Surface

Probes are synthesized onto each of the electrodes 108 by piezo inkjet printer with a printhead 201 containing multiple print nozzles 202 as seen in FIG. 2. The probe synthesis is as following process: (1) a droplet containing a chemical linker with a reactive thiol end is deposited onto a gold electrode at an indexed location. This process is also repeated on all the electrodes other indexed locations. (2) After a sufficient reaction time, the substrate is washed; and (3) a droplet containing a specific nucleotide, in some cases a modified nucleotide, or a nucleotide coupled to one or more redox molecules is deposited onto the linker functionalized electrode at the indexed location. This process is also repeated on all the electrodes at the other indexed locations. (4) After a sufficient reaction time, the substrate is washed. Steps (2) through (4) are repeated until the desired redox molecule labeled aptamer probes have been completely synthesized for each electrode at each indexed location on the substrate.

### Example 3: Probe Composition Example

A gold working electrode is functionalized with an aptamer probe, composed of an oligonucleotide sequence and labeled with a sequence of 3 redox molecules. The nucleotide sequence is attached to the gold surface of the working electrode by the reaction product of the linker 1-0-Dimethoxytrityl-hexyl disulfide, T-[(2-cyanoethyl)-(N,N-diisopropyl)]-phosphoramidite as seen in FIG. 7, thus linking the 3′ end of the oligonucleotide to the surface. The oligonucleotide sequence is a 25 mer nucleotide sequence including modified nucleotides. The 25 mer oligonucleotide has a sequence 5′-A-X¹-X²-X³-X⁴-X⁵-X⁶-X⁷-X⁸-X⁹-X¹⁰-X¹¹-X¹²-X¹³-X¹⁴-X¹⁵-X¹⁶-X¹⁷-X¹⁸-X¹⁹-X²⁰-X²¹-X²²-X²³-X²⁴-3′ where each of X¹-X²⁴ is independently any nucleotide or modified nucleotide and A is a nucleotide bound to three Methyl Blue redox molecules.

What is claimed is:

1. A biosensor device, comprising:
a. a substrate comprising one or more sensors;
b. one or more probes coupled to one or more sensors, wherein the one or more probes comprise:
i. an aptamer; and
ii. one or more redox molecules; and
c. an electrochemical circuit configured as an amperometric biosensor device; wherein
the substrate, the one or more probes and the electrochemical circuit comprise an integrated biosensors device.

2. The biosensor device of claim 1, wherein the substrate comprises a CMOS device.

3. The biosensor device of claim 1, wherein the one or more sensors comprise working electrodes.

4. The biosensor device of claim 1, wherein the aptamer comprises one or more nucleotides.

5. The biosensor device of claim 4, wherein the nucleotides comprise modified nucleotides.

6. The biosensor device of claim 1, wherein the aptamer specifically binds to the target.

7. The biosensor device of claim 1, wherein the electrochemical circuit, comprises the one or more working electrodes, one or more counter electrodes and a reference electrode; operably connected to a multipotentiostat; wherein the electrochemical circuit is configured for amperometric measurements.

8. The biosensor device of claim 2, wherein the CMOS device comprises a first working electrode of one or more working electrodes operably connected to a first transimpedance amplifier of one or more transimpedance amplifiers, wherein the transimpedance amplifier is operably connected to an analog-to-digital converter.

9. The biosensor device of claim 8, wherein the CMOS device comprises one or more analog-to-digital converters.

10. The biosensor device of claim 1, wherein the working electrodes comprise gold or hydrogenated amorphous carbon.

11. A method of detecting a target, using the biosensor device of claim 1, the method comprising:
a. contacting the one or more sensors with a sample comprising one or more targets;
b. changing the electrical surface potential of the one or more sensors thereby generating one or more electrical current signals corresponding to the one or more sensors; and
c. measuring the intensity of the one or more electrical current signals to detect the one or more targets.

12. The method of claim 11, wherein the one or more targets comprise small molecules.

13. The method of claim 11, wherein the one or more electrical current signals is generated by a change in the surface potential of a first working electrode of one or more working electrodes due to a change in distance between the one or more redox molecules of a first probe of the one or more probes and the first working electrode caused by a change in structure of the aptamer upon binding with a target of the one or more targets.

14. A method of synthesizing aptamer probes on a substrate, comprising:
a. providing a printer comprising a printhead, the printhead comprising one or more print nozzles;
b. providing a substrate;
c. disposing a droplet from a first print nozzle of the one or more print nozzles to a first indexed location of one or more indexed locations on the substrate;
d. replicating step (c) for a second print nozzle;
e. washing the substrate; and
f. repeating step (c) through (e) one or more times.

15. The method of claim 14, wherein the droplet comprises a nucleotide.

16. The method of claim 14, wherein the droplet comprises a modified nucleotide.

17. The method of claim 14, wherein the droplet comprises a redox molecule.

18. The method of claim 14, wherein the droplet comprises a linker molecule.

19. A probe composition having the formula:
[[A]ₙ[X]ₘ]_{y}-L-S, wherein;
a. each A independently comprises a monomer linked to one or more redox molecules;
b. each X independently comprises a monomer;
c. L comprises a linker;
d. S comprises a substrate;
e. each n is independently an integer from 0 to 10;
f. each m is independently an integer from 0 to 100; and
g. y is an integer from 1 to 10.

20. The probe composition of claim 19, wherein the monomer of one or more A or X comprises a nucleotide.

21. The probe composition of claim 20, wherein the nucleotide comprises a modified nucleotide.

22. The probe composition of claim 19, wherein the linker comprises a thiol functional group.

23. The probe composition of claim 19, wherein the substrate comprises at least one of gold, hydrogenated amorphous carbon or exposed OH groups.

24. The probe composition of claim 19, wherein the one or more redox molecules comprise Ferrocene.

25. The probe composition of claim 19, wherein the one or more redox labels comprise Methyl Blue.

26. The probe composition of claim 19, wherein the probe comprises at least 3 redox molecules.

27. The biosensor device of claim 1, wherein the electrochemical circuit, comprises the one or more working electrodes and one or more counter electrodes; operably connected to a multipotentiostat; wherein the electrochemical circuit is configured for amperometric measurements.

28. A biosensor device, comprising:
a substrate;
a buffer solution disposed over the substrate;
a working electrode attached to the substrate;
a probes electrically connected the working electrode, the probe comprising:
an aptamer attached to the substrate, the aptamer operable to bind to a target molecule, and
a redox molecule attached to the aptamer;
a counter electrode in electrical communication with the working electrode through a fluidic conductive path of the buffer solution;
a circuit configured for amperometric measurements that is electrically connected to the probe, the counter electrode and the working electrode;
wherein, when a target molecule binds to the aptamer, the aptamer is operable to change conformation, which results in a change of an electrical current that the circuit is operable to measure.

29. The biosensor device of claim 28, wherein the distance separating the working electrode from the counter electrode is within a range of about 500 micrometers to 3 millimeters.

30. The biosensor device of claim 29, wherein the biosensor device does not include a reference electrode.

31. The biosensor device of claim 28, wherein the substrate comprises exposed OH groups.

32. The biosensor device of claim 28, wherein the substrate comprises one of a CMOS device or a printed circuit board device.

33. The biosensor device of claim 28, comprising:
a lid encapsulating the substrate and the buffer solution, the lid including an inlet port and an outlet port that are operable to allow the buffer solution containing target molecules to pass therethrough;
wherein the counter electrode is positioned on an inner side of the lid.

34. The biosensor device of claim 33, comprising:
the working electrode comprising an array of working electrodes disposed on the substrate;
the probe comprising an array of probes, each probe connected to a working electrode of the array of working electrodes; and
the counter electrodes comprising one or more counter electrodes, each counter electrode being positioned within a distance of between about 500 micrometers to 3 millimeters of at least one working electrode of the array of working electrodes.

35. The biosensor device of claim 34, wherein the biosensor device does not include a reference electrode.

36. The biosensor device of claim 34, comprising:
an array of reference electrodes positioned on an outer side of the lid and extending therethrough to make contact with the buffer solution;

37. The biosensor device of claim 36, wherein each reference electrode of the array of reference electrodes is positioned within a distance of between about 500 micrometers to 3 millimeters of at least one working electrode of the array of working electrodes.

38. The biosensor device of claim 36, wherein the array of reference electrodes are disposed over an area that is at least 50 percent of the area that the working electrodes are disposed over.
